(19) United States
(12) Patent Application Publication
(10) Pub. No.: US 2021/0100872 A1
(43) Pub. Date: Apr. 8, 2021

(54) Pharmacological Formulation Comprising Cyclo (HIS-PRO) As Effective Ingredient For Preventing Or Treating Diabetes Mellitus

(71) Applicant: Novmetapharma Co., Ltd., Seoul (KR)

(72) Inventors: Heon Jong Lee, Incheon (KR); Kay Olmstead, Escondido, CA (US); Moon Ki Song, Northridge, CA (US); Kyong-Tai Kim, Gyeongsangbuk-do (KR); In-Kyu Lee, Daegu (KR); Hoe-Yune Jung, Gyeongsangbuk-do (KR)

(73) Assignee: Novmetapharma Co., Ltd., Seoul (KR)

(21) Appl. No.: 16/956,109

(22) PCT Filed: Dec. 11, 2018
(86) PCT No.: PCT/KR2018/015720

§ 371 Date: Jun. 19, 2020

(30) Foreign Application Priority Data
Dec. 20, 2017 (KR) 10-2017-0176322

(51) Int. Cl. A61K 38/12; A61K 33/30; A61P 3/10; A61K 9/48

(52) U.S. Cl. CPC A61K 38/12; A61K 9/48; A61P 3/10; A61K 33/30

(57) ABSTRACT

The present invention relates to a pharmaceutical formulation for preventing or treating diabetes mellitus, comprising cyclo-hispro as an active ingredient. Specifically, the present invention has the effect of lowering the concentration of glycated hemoglobin (HbA1c) in the blood, and further improving leptin resistance. In addition, the present invention relates to a pharmaceutical formulation for preventing or treating diabetes mellitus, characterized in that the pharmaceutical formulation comprises cyclo-hispro and zinc and is administered once a day.

### TECHNICAL FIELD

The present invention relates to a pharmaceutical formulation for preventing or treating diabetes mellitus, comprising cyclo-hispro as an active ingredient. Specifically, the present invention has the effect of lowering the concentration of glycated hemoglobin (HbA1c) in the blood, and further improving leptin resistance.

### BACKGROUND ART

Diabetes mellitus refers to the symptoms in which insulin, a glucose-regulating hormone secreted by the pancreatic beta cells, is not produced at the amount required by the body, or insulin fails to act properly in the cells, and thus glucose in the blood is not used as energy and accumulates in in the blood to cause hyperglycemia, and glucose is detected in the urine. In general, diabetes mellitus is classified into insulin-dependent diabetes mellitus (type 1 diabetes mellitus) and insulin-independent diabetes mellitus (type 2 diabetes mellitus) according to whether insulin is essentially required for the treatment of diabetes mellitus. Type 2 diabetes mellitus is an insulin-independent diabetes mellitus, which is caused by insufficient insulin action due to insulin resistance or relative lack of insulin. 90% of all patients with diabetes mellitus belong to type 2 diabetes mellitus, which is also referred to as adult-onset diabetes mellitus because it mostly develops after their 30s.

Insulin secretion disorder and insulin resistance are involved in the development of type 2 diabetes mellitus, and they are all defined by a plurality of genetic factors. In addition, environmental factors due to lifestyle are also mainly involved in insulin resistance. Insulin secretion disorder is caused by abnormalities in various genes related to insulin secretion ability. In addition, insulin resistance is increased by abnormalities in various genes related to insulin sensitivity. Hyperglycemia is caused by insufficient insulin action as well as environmental factors such as obesity, overeating, high fat diet, lack of exercise, stress, and increased age, leading to the development of type 2 diabetes mellitus.

In addition, it has been known from a long time ago that obesity is a risk factor for the development of type 2 diabetes mellitus. Recently, a result of study has been published showing that there is a direct relevance between the development of type 2 diabetes mellitus and obesity. It is known that obesity and type 2 diabetes mellitus are very closely associated with insulin resistance. When insulin resistance occurs in the body, insulin function that lowers blood glucose level is decreased, leading to problems in maintaining adequate blood glucose level. A result of study has been reported showing that obese adults have a seven-fold higher chance of developing diabetes mellitus compared to adults with normal body weight.

In order to reduce the occurrence of complications that occur in patients treated for diabetes mellitus, it is important to maintain the blood glucose levels at appropriate levels. Since the blood glucose level measured at one time point can change due to various factors, glycated hemoglobin (HbA1c) test is a test that is most widely used for the purpose of determining the pattern of long-term glycemic control. The glycosylated hemoglobin is in the form in which glucose is bound to hemoglobin normally present in red blood cells, and when the blood glucose level is maintained high, the level of glycated hemoglobin is also increased. Since glycated hemoglobin reflects the average blood glucose level for 2 to 4 months, it is useful for determining the pattern of long-term glycemic control.

The American Diabetes Association (ADA) has generally set the target level for glycated hemoglobin to less than 7% since 2012. However, the American Diabetes Association (ADA) has recommended that the target level is regulated more strictly at 6.0 to 6.5%, when the patient's willingness and effort for treatment are high, the risk of hypoglycemia is low, the disease period of diabetes mellitus is short, the life expectancy is long, and there are no comorbidities and vascular complications. In the opposite case, the

American Diabetes Association (ADA) has recommended that the target level is regulated at 7.5 to 8% and diabetes mellitus is treated differently depending on the characteristics of the patient. An increase in glycated hemoglobin of 1% is equivalent to an average increase in blood glucose of 35 mg/dL. Therefore, the present inventors tried to provide a fundamental method for treating diabetes mellitus by developing a method for preventing or treating diabetes mellitus, which can obtain an effect of reducing the level of glycated hemoglobin.

If diabetes mellitus persists for a long period of time, the absorption of glucose into the body does not normally occur, leading to abnormalities in glucose metabolism, lipid metabolism, and protein metabolism. As a result, several complications of diabetes mellitus develop including hyperinsulinemia, nerve complications, diabetic retinopathy (non-proliferative retinopathy, proliferative retinopathy, diabetic cataract), renal failure, sexual dysfunction, skin disease (allergy), hypertension, atherosclerosis, stroke (apoplexy), heart disease (myocardial infarction, angina, heart attack). Therefore, in order to understand various causes and etiologies of type 2 diabetes mellitus and to provide measures for improvement, studies on glucose transport and metabolic processes, insulin signaling systems and the like have been actively conducted at home and abroad. However, there is still no development of a drug that can treat diabetes mellitus fundamentally.

Korean Patent Laid-Open Publication No. 2001-0022786 discloses a composition comprising zinc ion and cyclo-hispro as a composition for treating diabetes mellitus in mammals. However, the above invention is merely to confirm the change in glucose concentration by administering a composition of zinc ion and cyclo-hispro to the animal, and has not been confirmed whether it has a pharmacological effect to the extent in which it can be applied to the human body to prevent or treat the disease and its effective content.

One of the most important tasks in drug administration is to find a dosage and a method of administration that have few side effects and are consistently efficacious. It is very complicated to find the optimal dosage due to the serum half-life, the relationship between dosage and efficacy, especially the correlation with other therapeutic agents for diabetes mellitus that are previously prescribed and the like. In addition, in the case of therapeutic agent for diabetes mellitus, which is a drug that must be taken for a long period of time, the method of administration is also very important. In the case of a formulation that is administered parenterally such as an injection or administered several times a day, the patient's compliance with medication is low, and it is difficult to treat diabetes mellitus and prevent complications thereof through an effective medication.

### DETAILED DESCRIPTION OF THE INVENTION

### Technical Problem

The present invention is to provide a pharmaceutical formulation for preventing or treating diabetes mellitus, comprising cyclo-hispro or a pharmaceutically acceptable salt thereof, which exhibits few or less side effect even when taken for a long period of time. In particular, the present invention is to provide an effective method for preventing and treating diabetes mellitus by providing a dosage and a method of administration of cyclo-hispro or a pharmaceutically acceptable salt thereof that reduces the level of glycated hemoglobin (HbA1c) and improves insulin resistance and leptin resistance.

### Solution to Problem

In order to achieve the above object, the present invention provides a pharmaceutical formulation for preventing or treating diabetes mellitus, characterized in that the pharmaceutical formulation comprises cyclo-hispro or a pharmaceutically acceptable salt thereof as an active ingredient and is administered at an amount of 1 mg to 25 mg per day.

“Cyclo-hispro (cyclo(his-pro))” is a naturally occurring cyclic dipeptide that has a structure of formula I below and is composed of histidyl and proline.

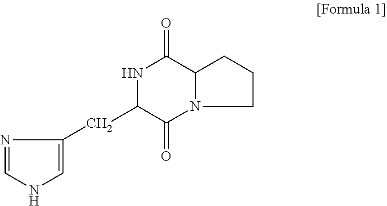 [SMILES: O=C1NC(Cc2c[nH]cn2)C(=O)N2CCCC12]

In particular, the diabetes mellitus may be type 2 diabetes mellitus, and the formulation may be administered orally once a day.

In addition, the cyclo-hispro or pharmaceutically acceptable salt thereof may be administered at an amount of 3 mg to 20 mg per day, preferably may be administered at an amount of 6 mg to 15 mg per day, and most preferably may be administered at an amount of 15 mg per day.

The present invention provides a pharmaceutical formulation for preventing or treating diabetes mellitus, comprising cyclo-hispro or a pharmaceutically acceptable salt thereof and zinc as active ingredients.

Specifically, the daily dose of cyclo-hispro or a pharmaceutically acceptable salt thereof may be 1 mg to 25 mg, and the daily dose of zinc may be 15 mg to 30 mg.

The present invention provides an effect of preventing or treating diabetes mellitus by reducing the concentration of glycated hemoglobin (HbA1c) in the blood over a long period of time.

### Effect of Invention

The pharmaceutical formulation of the present invention comprises cyclo-hispro or a pharmaceutically acceptable salt thereof as an active ingredient, and thus the present invention provides a method for preventing or treating diabetes mellitus having few side effects caused by a drug even when taken for a long period of time. In addition, by reducing the concentration of glycated hemoglobin (HbA1c) in the blood over a long period of time, it is possible to substantially improve and treat symptoms of diabetes mellitus and complications thereof.

In addition, the pharmaceutical formulation of the present invention comprises cyclo-hispro or a pharmaceutically acceptable salt thereof, and the present invention provides an effective administration method and dosage for the prevention and treatment of diabetes mellitus.

### BRIEF DESCRIPTION OF DRAWINGS

FIG. 1 shows the change in concentration (%) of HbA1c over time over 12 weeks for each administration group.

FIG. 2 shows the change in body weight (kg) over time over 12 weeks for each administration group.

### BEST MODE FOR CARRYING OUT THE INVENTION

The present inventors confirmed that when cyclo-hispro or a pharmaceutically acceptable salt thereof was administered in a fixed dose, the effect of preventing or treating diabetes mellitus was remarkably increased. Based on the above, the present inventors completed the present invention.

The present invention provides a pharmaceutical formulation for preventing or treating diabetes mellitus, characterized in that the pharmaceutical formulation comprises cyclo-hispro or a pharmaceutically acceptable salt thereof as an active ingredient and is administered at an amount of 1 mg to 25 mg per day.

“Cyclo-hispro (cyclo(his-pro))” is a naturally occurring cyclic dipeptide that has a structure of formula I below and is composed of histidyl and proline.

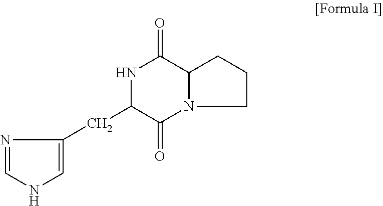 [SMILES: O=C1NC(Cc2c[nH]cn2)C(=O)N2CCCC12]

Cyclo-hispro is found in blood, cerebrospinal fluid (CSF), semen, brain, spinal cord, and gastrointestinal tract of human, and the like. Cyclo-hispro has several biological activities. According to studies, it was found that cyclo-hispro is one of the major metabolites of thyrotropin-releasing hormone (TRH), which is the most produced in the prostate.

“Cyclo-hispro” of the present invention may include purified cyclo-hispro.

In addition, the cyclo-hispro or pharmaceutically acceptable salt thereof may be administered at an amount of 3 mg to 20 mg per day, preferably may be administered at an amount of 6 mg to 15 mg per day, and most preferably may be administered at an amount of 15 mg per day.

The “diabetes mellitus” may be type 1 or type 2 diabetes mellitus, and preferably may be type 2 diabetes mellitus.

The pharmaceutical formulation of the present invention may be administered once a day or several times a day, and preferably may be administered once a day.

The pharmaceutical formulation of the present invention may be orally or parenterally administered, and preferably may be orally administered.

The pharmaceutical formulation of the present invention may be orally administered to an individual via various routes. All methods of administration may be used, and the pharmaceutical formulation of the present invention may be administered, for example, by oral administration, intranasal administration, transbronchial administration, intraarterial injection, intravenous injection, subcutaneous injection, intramuscular injection, or intraperitoneal injection. Preferably, the pharmaceutical formulation of the present invention may be administered orally once a day.

The present invention provides a pharmaceutical formulation for preventing or treating diabetes mellitus by reducing the concentration of HbA1c in the blood for a long period of time.

The glycated hemoglobin (HbA1c) test reflects the changes in blood glucose during the previous 2 to 3 months. According to several studies, it was found that an increase in glycated hemoglobin of 1% is equivalent to an average increase in blood glucose of 35 mg/dL. That is, the glycated hemoglobin is an indicator of long-term treatment of diabetes mellitus, and it can be confirmed whether continuous relief and improvement of symptoms of diabetes mellitus are achieved, and the present invention has an effect of reducing glycated hemoglobin.

The present invention provides a pharmaceutical formulation for preventing or treating diabetes mellitus, comprising cyclo-hispro or a pharmaceutically acceptable salt thereof and zinc as active ingredients.

The term “zinc” means to include all zinc salts, zinc ions, zinc cations, and zinc anions.

Specifically, the daily dose of cyclo-hispro or a pharmaceutically acceptable salt thereof may be 1 mg to 25 mg, and the daily dose of zinc may be 15 mg to 30 mg.

In addition, the daily dose of the zinc may be preferably 23 mg.

The present invention relates to a pharmaceutical formulation for preventing or treating diabetes mellitus, characterized in that the pharmaceutical formulation comprises 15 mg of cyclo-hispro and 23 mg of zinc and is administered once a day.

The term “pharmaceutically acceptable salt” means a salt that is commonly used in the pharmaceutical field, including hydrochloride, hydrobromide, hydroiodide, hydrofluoride, sulfate, sulfonate, citrate, camphorate, maleate, acetate, lactate, nicotinate, nitrate, succinate, phosphate, malonate, malate, salicylate, phenyl acetate, stearate, formate, fumarate, urea, sodium, potassium, calcium, magnesium, zinc, lithium, cinnamate, methylamino, methanesulfonate, picolinate, p-toluenesulfonate, naphthalenesulfonate, tartrate, triethylamino, dimethylamino, and tri(hydroxymethyl)aminomethane, but not limited thereto.

In order to prepare the pharmaceutical formulation of the present invention, appropriate carriers, excipients, and diluents that are commonly used may be further included.

In addition, it may be formulated and used in the form of oral preparation such as powders, granules, tablets, capsules, suspensions, emulsions, syrups, and aerosols, external preparations, suppositories, and sterile injectable solutions according to a conventional method.

Carriers, excipients, and diluents that may be included in the formulation include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil, and the like, but are not limited thereto.

In addition, the formulation may further comprise diluents or excipients such as conventional fillers, bulking agents, binders, wetting agents, disintegrating agents, surfactants, and the like.

Hereinafter, preferred examples are presented in order to help the understanding of the present invention. However, the following examples are only provided to more easily understand the present invention, and the contents of the present invention are not limited by the following examples.

### EXAMPLE

We conducted a clinical trial designed as randomized, placebo-controlled, double-blind, and two-way crossover on drug interaction in patients with type 2 diabetes mellitus. The clinical trial was conducted for 149 patients over a total of 16 weeks, in which the screening period was 2 weeks, the treatment period was 12 weeks, and the period of the evaluation of safety and follow-up was 2 weeks.

In the screening stage, 64 subjects were selected from 149 participants, and the subjects who met the selection criteria were fasted for 2 hours before and after breakfast each day and subjected to a 2-week evaluation process of measuring and recording blood glucose levels. In the treatment stage, capsules containing placebo or CHP with different doses were randomly administered to 64 selected subjects once a day for 12 consecutive weeks.

In the above experiment, a capsule containing CHP (cyclo-hispro) with a different dose or a placebo corresponding thereto was used, and each subject received a two-week dose (18 capsules =14 +4) at one visit and was instructed to take 1 capsule before bedtime every day. In this case, the anti-diabetic drugs and other prescribed drugs currently being used were continuously taken. Each subject was assigned at the same rate as follows and administered with a placebo or a compound with a different dose, respectively.
Administration Group A: 3 mg of CHP+23 mg of zinc—16 subjects
Administration Group B: 9 mg of CHP+23 mg of zinc—16 subjects
Administration Group C: 15 mg of CHP+23 mg of zinc—16 subjects
Administration Group D: placebo (control)—16 subjects

### Example 1

### Experiment of Change in Concentration of HbA1c for Each Administration Group for Different Dose of CHP

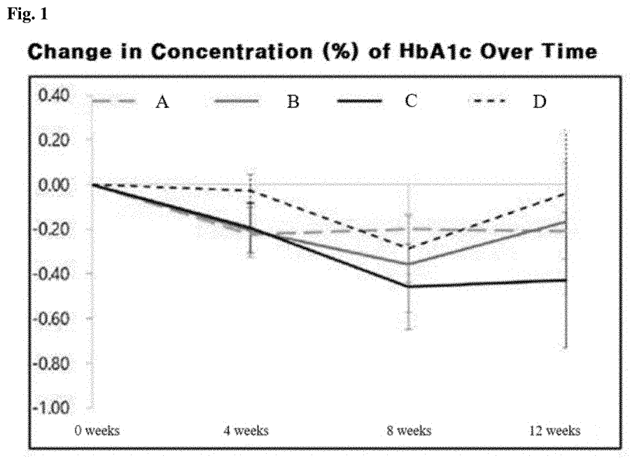

The present inventors observed the change in concentration of HbA1c during the 12-week dosing period. Since the blood glucose level measured at one time point can change due to various factors, the change in glycated hemoglobin (HbA1c) was checked for the purpose of determining the pattern of long-term glycemic control, and the results are shown in Table 1 and FIG. 1.

TABLE 1

| | | Administration | Administration | Administration | Administration |
|---|---|---|---|---|---|
| | | Group A | Group B | Group C | Group D |
| Baseline | Number of | 15 | 16 | 16 | 14 |
| (Week 0) | Subjects | | | | |
| | Average | 8.25 | 8.06 | 8.49 | 8.29 |
| Week 12 | Number of | 12 | 15 | 14 | 13 |
| | Subjects | | | | |
| | Average | 8.04 | 7.90 | 8.08 | 8.21 |
| Change Value | Number of | 12 | 15 | 14 | 13 |
| (From Week 0 | Subjects | | | | |
| to Week 12) | | | | | |
| | Average | −0.21 | −0.17 | −0.43 | −0.04 |

As shown in Table 1 above, the level of HbA1c at week 12 in Administration Group C was −0.43%, which was the lowest concentration, and the levels of HbA1c at week 12 in Administration Group A and Administration Group B were lower than that of Administration Group D (control).

In addition, as shown in FIG. 1, the level of HbA1c over time was reduced overall in all groups between week 0 and week 4, and in Administration Group C, the level of HbA1c was reduced consistently until week 8, and then there was little change in the level of HbA1c between week 8 and week 12.

As a result, it was confirmed that the level of HbA1c in all patient groups receiving CHP was reduced compared to the control.

### Example 2

### Comparative Experiment of Change in Body Weight for Each Administration Group for Different Dose of CHP

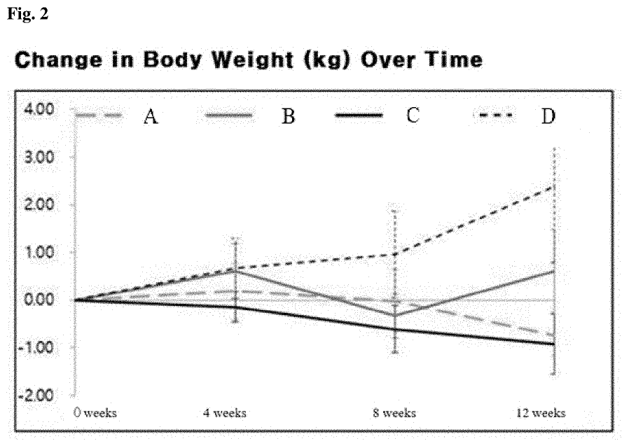

The present inventors observed the change in body weight of subjects through the 12-week experiment, and the results are shown in Table 2 and FIG. 2.

TABLE 2

| | | Administration | Administration | Administration | Administration |
|---|---|---|---|---|---|
| | | Group A | Group B | Group C | Group D |
| Baseline | Number of | 15 | 16 | 16 | 14 |
| (Week 0) | Subjects | | | | |
| | Average | 109.11 | 103.56 | 103.94 | 109.61 |
| Week 12 | Number of | 12 | 15 | 14 | 13 |
| | Subjects | | | | |
| | Average | 108.28 | 105.19 | 104.12 | 113.43 |
| Change Value | Number of | 12 | 15 | 14 | 13 |
| (From Week 0 | Subjects | | | | |
| to Week 12) | | | | | |
| | Average | −0.73 | 0.61 | −0.92 | 2.38 |

As shown in Table 2, it was confirmed that the body weight at week 12 was increased the most (+2.38 kg) in Administration Group D. On the other hand, it was confirmed that the body weight at week 12 was greatly reduced (—0.92 kg) in Administration

Group C. The weight loss of Administration Group A (−0.73 kg) was less than that of Administration Group C, but the body weight was reduced. It was confirmed that the weight gain of Administration Group B (+0.61 kg) was not greater than that of Administration Group D (control).

In addition, as shown in FIG. 2, it was confirmed that the body weight of Administration Group D was increased consistently until week 12, and the body weight of Administration Group C was reduced consistently until week 12.

As a result, it was confirmed that the weight gain in all patient groups receiving CHP was less than the control, or the body weight was reduced.

### Example 3

### Comparative Experiment of Improvement of Body Mass Index (BMI) and Leptin Resistance for Each Administration Group for Different Dose of CHP

The present inventors confirmed the improvement of body mass index (BMI) and the improvement of leptin resistance, and the results are shown in Tables 3 and 4.

Leptin is an appetite suppressing protein secreted from adipose cells, and is known as a hormone to act on the brain after being secreted from adipose tissue to suppress appetite and activate metabolism in the body, thereby reducing body weight. In the case of humans, the more obese people (the more adipose tissue), the higher the concentration of leptin in the blood. This indicates the resistance to leptin action.

TABLE 3

| | | Administration | Administration | Administration | Administration |
|---|---|---|---|---|---|
| | | Group A | Group B | Group C | Group D |
| Baseline | Number of | 15 | 16 | 16 | 14 |
| (Week 0) | Subjects | | | | |
| | Average | 37.18 | 36.29 | 37.23 | 37.91 |
| Week 12 | Number of | 12 | 15 | 14 | 13 |
| | Subjects | | | | |
| | Average | 36.45 | 36.59 | 37.48 | 38.72 |
| Change Value | Number of | 12 | 15 | 14 | 13 |
| (From Week 0 | Subjects | | | | |
| to Week 12) | | | | | |
| | Average | −0.27 | 0.16 | −0.28 | 0.66 |

As shown in Table 3, it was confirmed that the body mass index (BMI) at week 12 was reduced by −0.27 kg/m² and −0.28 kg/m² in Administration Group A and Administration Group C, respectively, but the body mass index (BMI) was increased by 0.66 kg/m² in Administration Group D.

TABLE 4

| | | Administration | Administration | Administration | Administration |
|---|---|---|---|---|---|
| | | Group A | Group B | Group C | Group D |
| Baseline | Number of | 15 | 16 | 16 | 14 |
| (Week 0) | Subjects | | | | |
| | Average | 10.93 | 19.85 | 12.93 | 13.58 |
| Week 12 | Number of | 12 | 15 | 14 | 12 |
| | Subjects | | | | |
| | Average | 9.66 | 9.90 | 10.59 | 11.33 |
| Change Value | Number of | 12 | 15 | 14 | 12 |
| (From Week 0 | Subjects | | | | |
| to Week 12) | | | | | |
| | Average | 0.59 | −3.42 | −3.39 | −1.87 |

In addition, as shown in Table 4, it was confirmed that the plasma leptin level at week 12 was reduced to a statistically significant level in Administration Group B and Administration Group C compared to the reference value. As a result, it was confirmed that both the body mass index and plasma leptin level were reduced in Administration Group C.

### Example 4

### Result of Comparative Experiment of Evaluation of Safety for Each Dose of Compound 1

The present inventors conducted the evaluation of safety. As a result, mild and moderate side effects that are not related to the CHP were observed, and side effects of increased blood glucose level, dizziness, and ecchymosis were more frequently observed in the control receiving placebo.

As a result, it can be seen that when the CHP administration groups are compared with the control (placebo), the CHP has an excellent effect as a therapeutic agent for diabetes mellitus and has additional advantages such as reduced body weight, reduced leptin level, and reduced side effects.

1. A pharmaceutical formulation for preventing or treating diabetes mellitus, characterized in that the pharmaceutical formulation comprises cyclo-hispro or a pharmaceutically acceptable salt thereof as an active ingredient and is administered at an amount of 1 mg to 25 mg per day.

2. The pharmaceutical formulation according to claim 1, characterized in that the diabetes mellitus is type 2 diabetes mellitus.

3. The pharmaceutical formulation according to claim 1, characterized in that the formulation is administered orally once a day.

4. The pharmaceutical formulation according to claim 1, characterized in that the cyclo-hispro or pharmaceutically acceptable salt thereof is administered at an amount of 3 mg to 20 mg per day.

5. The pharmaceutical formulation according to claim 4, characterized in that the cyclo-hispro or pharmaceutically acceptable salt thereof is administered at an amount of 6 mg to 15 mg per day.

6. The pharmaceutical formulation according to claim 5, characterized in that the cyclo-hispro or pharmaceutically acceptable salt thereof is administered at an amount of 15 mg per day.

7. The pharmaceutical formulation according to claim 1, characterized in that the formulation lowers the concentration of HbA1c in the blood.

8. The pharmaceutical formulation according to claim 1, characterized in that the pharmaceutical formulation further comprises zinc.

9. The pharmaceutical formulation according to claim 8, characterized in that the zinc is administered at an amount of 15 mg to 30 mg per day.

10. The pharmaceutical formulation according to claim 9, characterized in that the zinc is administered at an amount of 23 mg per day.

11. A pharmaceutical formulation for preventing or treating diabetes mellitus, characterized in that the pharmaceutical formulation comprises 15 mg of cyclo-hispro and 23 mg of zinc and is administered once a day.
